(19) United States
(12) Patent Application Publication
(10) Pub. No.: US 2009/0148927 A1
(43) Pub. Date: Jun. 11, 2009

(54) Mass Production Of Aquatic Plants

(75) Inventors: Del C. Schroeder, Warren, MI (US); Robert W. Truxell, Bloomfield Hills, MI (US); Michael J. Bartus, Clawson, MI (US)

Correspondence Address: HARNESS, DICKEY & PIERCE, P.L.C., P.O. BOX 828, BLOOMFIELD HILLS, MI, 48303, US

(73) Assignee: SEQUEST, LLC, Bloomfield Hills, MI (US)

(21) Appl. No.: 12/327,178

(22) Filed: Dec. 3, 2008

Related U.S. Application Data
(60) Provisional application No. 61/005,472, filed on Dec. 5, 2007.

(51) Int. Cl. C12N 1/12; C12M 1/00

(52) U.S. Cl. 435/257.1; 435/292.1

(57) ABSTRACT

A method of mass production of algae is provided, including an algae growth trough having an algae introduction end having a first width and an algae extraction end having a second width wider than the first width. Water is supplied to the trough from a water treatment facility and carbon dioxide is introduced to the water from a combustion source. The algae is allowed to grow within the algae growth trough while light is provided to the trough. The algae is extracted from the extraction end of the trough, and as the algae continues to grow under optimal conditions, the algae is continuously harvested for commercial use. The mass production of algae allows for the consumption of enormous amounts of carbon dioxide which can be generated from a coal electrical generation facility or other industrial facility.

### CROSS-REFERENCE TO RELATED APPLICATIONS

This application claims the benefit of U.S. Provisional Application No. 61/005,472, filed on Dec. 5, 2007, the disclosure of which is incorporated herein by reference.

### FIELD

The present disclosure relates to a system for the mass production of aquatic plants and more particularly, to a system that can utilize water from a waste management plant as well as carbon dioxide from a power generation plant or other industrial plant to enhance the mass production of aquatic plants such as algae.

### BACKGROUND

The statements in this section merely provide background information related to the present disclosure and may not constitute prior art.

The emissions of carbon dioxide emanating from the burning of fossil fuels such as coal is increasingly on the minds of environmentalists, the general public and now our lawmakers. Pressures from around the globe will demand that solutions be applied to the problem of carbon dioxide emissions. Certain states within the United States have taken the lead in passing their own legislation on carbon dioxide emission standards and it is expected that the U.S. Congress will implement a national policy before too long. As a result, carbon dioxide emissions will progress from a nuisance to a liability and soon, corporate America will be in a dire need for a solution to the problem of carbon dioxide emissions.

At present, the most popular method being considered is geological sequestration of carbon dioxide gasses. This plan involves injecting carbon dioxide directly into underground geological formations. Not only is this method expensive, it is still unproven that buried carbon dioxide gasses will remain buried. Companies are earmarking countless millions on the technology because they see no other alternative. A significant number of people believe that man is releasing more carbon dioxide gasses into our atmosphere than our eco system can handle, thus damaging our planet's ability to maintain a balanced environment. Due to the combustion of fossil fuels and deforestation, the concentration of atmospheric carbon dioxide has increased dramatically since the beginning of the age of industrialization. The number one source for the release of carbon dioxide occurs during electrical generation, specifically, from coal-fired power plants. Some 37% of all carbon dioxide emissions from energy producers and industry come from the burning of coal.

Although carbon dioxide gas is not yet regulated at the federal level in the United States, many states have set their own rules and experts agree, it is just a matter of time when unrestricted release of carbon dioxide gasses will be prohibited. The European Union has been proactive in setting emission standards. Business entities are allocated emission allowances every year. The European Union scheme allows a regulated operator to use carbon credits in the form of emission reduction units to comply with its obligations. Those exceeding their limits are penalized and unused credits can be traded between entities. The current rate for carbon dioxide emissions is $38 per ton. Here in the United States, a volunteer but legally binding emissions trading market has begun. Although carbon dioxide is currently trading at over $3 per ton, a recent analysis at MIT reports that the expected move by congress will skyrocket the price of carbon dioxide emissions. Under several different possible actions that may be taken by lawmakers, the price is likely to reach from $18 to $55 per ton by 2015 and from $30 to $200 per ton by 2050. These costs are ultimately going to be passed onto the consumers. It is expected that lawmakers will soon set carbon dioxide emission regulations and thus create massive liabilities for emitters. In 2006, the United State's consumption of coal resulted in the release of 2.3 billion tons of carbon dioxide.

Algae are one of the simplest organisms known to man. In nature, algae consume carbon dioxide through the photosynthesis process, and produces about 70% of the global production of oxygen on earth. Photosynthesis is the conversion of light energy into chemical energy by living organisms. Some of the basic elements algae need to survive include carbon dioxide and water, together with sunlight as its energy source to make an end product that consists of protein, carbohydrates, fatty acids, lipids and oxygen. All of these are marketable byproducts as a result of growing and harvesting the algae. Algae can be used to make biodiesel, bioethanol, and biobutanol and by some estimates can produce vastly superior amounts of vegetable oil, compared to terrestrial crops grown for the same purpose. Algae can be grown to produce biomass, which can be burned to produce heat and electricity. Algae are commonly used in waste water treatment facilities, reducing the need for greater amounts of toxic chemicals that are already used. Furthermore, algae bioreactors are used by some power plants to reduce carbon dioxide emissions. The carbon dioxide can be pumped into a pond or some kind of tank, on which the algae feed. Alternatively, the bioreactor can be installed directly on top of a smoke stack. The utilization of carbon dioxide emissions from power plants and other industrial plants can reduce the carbon dioxide credits utilized by these industries. United States coal-fired utilities produce over two billion tons of carbon dioxide annually and to the extent that this carbon dioxide can be utilized in a beneficial manner to provide environmental and financial benefit, a mass production facility for producing algae and consuming carbon dioxide emissions has huge potential. Algae's potential for production of bio fuels such as biodiesel, bioethanol and biobutanol as well as its potential for petro chemical feed stocks has great potential. In addition, the use of algae as a food additive for human consumption, livestock feed, fish farming industry and aquarium enthusiasts as fish food also has great potential. Furthermore, within the agriculture industry, the algae biomass can serve as a fertilizer and reduce the use of petroleum chemical fertilizers in the process. Algae can also be combined with waste water treatment plant waste cake material, to provide additional BTU value. This additional nutrient BTU content of the waste cake material enhances the value of algae for either a renewable fuel source or for it's fertilizer nutrient value. Furthermore, there is potential use for algae byproducts in consumer products such as in the pharmaceutical industry and cosmetic industry. Finally, in the manufacturing industry, algae plant fibers can be used for paper products as an excellent method to utilize the remaining algae mass and save trees in the process.

Although algae has grown naturally in lakes and in ponds throughout the world, and it has been grown in small batch processes under controlled conditions, there is a need for a system of mass production of algae.

### SUMMARY

A method of mass production of algae or other aquatic plants is provided including providing a plant growth trough having an introduction end having a first width and an extraction end having a second width wider than the first width. Nutrient rich UV or Gamma Ray sterilized water is provided to the trough and can be provided from a water treatment facility or other water supply source. Carbon dioxide is introduced to the water from a combustion source that can include an electricity generation plant that burns coal or other fossil fuels or from an industrial manufacturing plant that uses combustion processes in the manufacturing of materials such as steel, cement, and coke operations. A source of light is provided to the trough and can include natural sunlight, LED lights, florescent and incandescent lights as well as other available lighting sources.

Algae or other aquatic plants are introduced to the trough at the introduction end. As the algae grows it is allowed to expand through an increasingly widening trough with the algae being extracted from the extraction end of the trough. The trough can be fully enclosed to maintain ideal growing conditions and to reduce foreign contaminants. Also a fully enclosed trough enables the captured Oxygen that is given off from the algae growth as the carbon is absorbed in the photosynthesis process. The water from a waste treatment plant can provide all of the water and nutrients required to create the optimal growing environment. The algae can be selected from the most productive strains of algae for maintaining carbon dioxide absorption and product output. A controlled carbon dioxide/air/water/nutrient mixture will constantly flow throughout the troughs to maximize algae growth.

In order to absorb the massive amounts of carbon dioxide gasses that can be generated from an electrical generation plant or other industrial facility, the algae production facility can be arranged with troughs that are stacked from floor to ceiling and with troughs arranged in strategic patterns to facilitate mass production and harvesting of the algae. The troughs can be made at least in part from a polyethylene plastic which is resistant to the algae connecting thereto. The troughs can include a plurality of straight sections, each having a generally constant width and attached to transition sections that widen from one end to another. Each of the straight sections can have a same width with the transition section diverting flow to two adjacent straight sections.

The straight sections can be made from extruded plastic and can be reinforced by extruded metal members which are engaged with the extruded plastic sections. The straight sections can be assembled from a first side panel including a side wall portion and a first floor portion and a second side panel including a side wall portion and a second floor portion. The first floor portion and the second floor portion can each include mutual engaging portions for sealingly engaging the first and second side panels together. Alternatively, the straight sections can include a center floor section including first and second edge portions adapted to engage respective ones of the first floor portion and the second floor portion for providing straight sections of varying widths. The components of the straight sections can be welded together to provide a sealed connection there between. The algae growth facility can include multiple floor levels with multiple trough layers per floor and can further include wind generators and solar panels on the walls and roof of the facility to potentially add power that can be utilized in the facility to power the light sources and pumps for the algae growth.

Further areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way.

FIG. 1 is a schematic diagram of the process for mass production of algae according to the principles of the present invention;

FIG. 2 is a schematic diagram of an alternative process for the mass production of algae according to the principles of the present invention;

FIG. 3 is a schematic diagram of a further embodiment of the process for the mass production of algae in combination with a coal liquefaction plant according to the principles of the present disclosure;

FIG. 4 is a cross-sectional view of a trough used for the mass production of algae according to the principles of the present disclosure;

FIG. 4a is a perspective view of a trough insert used for the mass production of algae according to the principles of the present disclosure;

FIG. 4b is a cross-sectional view of a light strip incorporated into the trough insert used for the mass production of algae according to the principles of the present disclosure;

FIG. 5 is a top plan view of an exemplary trough utilized for the mass production of algae according to the principles of the present disclosure;

FIG. 6 is a partial cross-sectional view of a side panel used for constructing the trough of FIG. 5;

FIG. 7 is a perspective view illustrating the assembly of a trough such as disclosed in FIG. 5;

FIG. 7a is a perspective view of a trough having well portions from which water can be extracted;

FIG. 8 is an exploded schematic view illustrating the assembly of the trough sections of FIG. 7;

FIG. 9 is a partial cross-sectional view illustrating the engagement between trough sections according to the principles of the present disclosure;

FIG. 10 is a plan view of an alternative trough construction according to the principles of the present disclosure;

FIG. 10a illustrates a diverter section according to the principles of the present disclosure;

FIG. 10b is a plan view of a straight trough section according to the principles of the present disclosure;

FIGS. 10c-10e illustrate various diverter sections that can be utilized for assembling the trough of FIG. 10;

FIG. 10f is an end plate used in the construction of the trough structure shown in FIG. 10;

FIG. 11 is a schematic view of the layout of the algae troughs in an algae growth facility according to the principles of the present disclosure;

FIG. 12 illustrates an alternative facility layout for the mass production of algae;

FIG. 13 is still a further alternative layout of the algae growth facility according to the principles of the present disclosure;

FIG. 14 is a side plan view of the algae production facility shown in FIG. 11;

FIG. 15 is a schematic illustration of the stacked troughs in a three floor facility for mass production of algae according to the principles of the present disclosure;

FIG. 16 illustrates an alternative arrangement of the algae production troughs according to the principles of the present disclosure;

FIG. 17 illustrates a further alternative arrangement of the algae production troughs according to the principles of the present disclosure;

FIG. 18 shows an alternative self-feeding trough arrangement where algae is extracted from the extraction end of the trough and introduced into an introduction end in a continuous loop in order to reduce the amount of work associated with reintroducing algae into the growth trough;

FIG. 19A is a perspective view of a conveyor matt used within the troughs for harvesting bentic algae according to the principles of the present disclosure; and

FIG. 19B is a schematic view of the conveyor matt of FIG. 19A within a trough for harvesting algae according to the principles of the present disclosure.

### DETAILED DESCRIPTION

The following description is merely exemplary in nature and is not intended to limit the present disclosure, application, or uses.

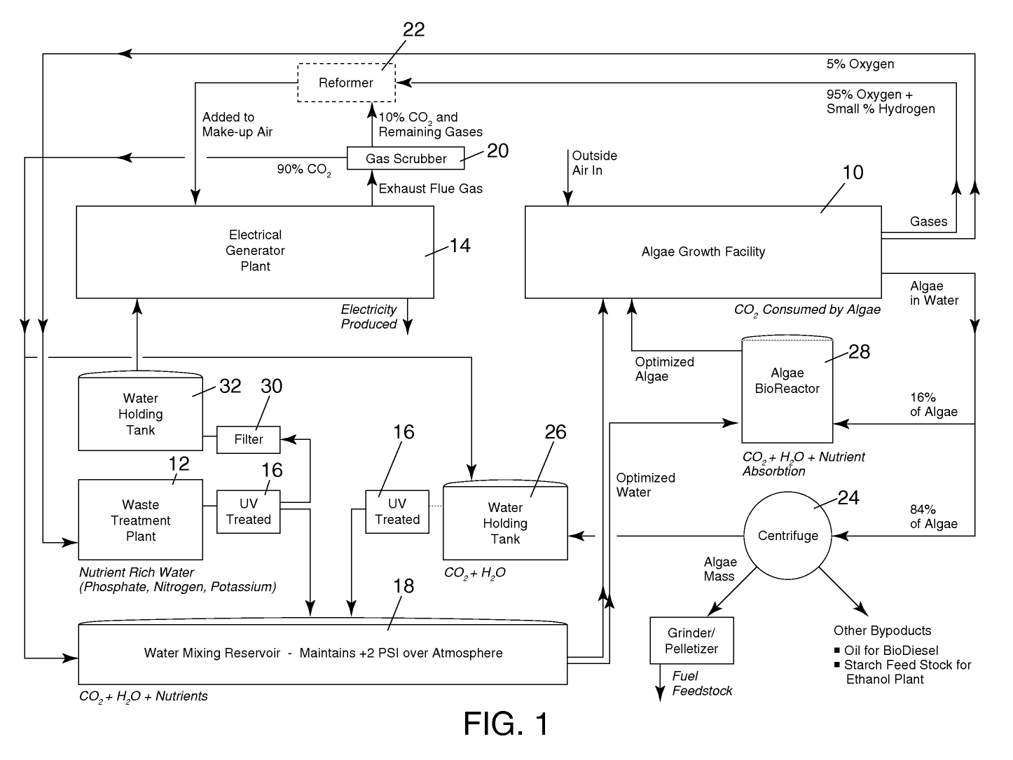

With reference to FIG. 1, a method and system for the mass production of algae will now be described. The system includes an algae growth facility 10 which is supplied with large amounts of water from a waste treatment plant 12 and is supplied with carbon dioxide from an electrical generator plant 14 or other industrial facility that utilizes the combustion of fossil fuels that create carbon dioxide as a byproduct. Thus, the algae growth facility 10 is provided with nutrient rich water and abundant amounts of carbon dioxide to facilitate to the mass production of algae that can then be used for other products such as biodiesel, bioethanol and other consumer products.

As shown in the process diagram of FIG. 1 the waste treatment plant 12 provides nutrient rich water (including phosphates, nitrogen, and potassium) which can be treated with ultraviolet light by a UV treatment device 16 that essentially kills the bacteria in the water by the exposure to ultraviolet light. The UV treatment device 16 can be of the type available from Atlantic Ultraviolet Corporation, 375 Marcus Blvd. Hauppauge, N.Y. 11788. The UV treatment device 16 kills the bacteria but allows the nutrients to stay in the water without the added necessity of adding chemical treatments that could inhibit algae growth. The UV treated water from the UV treatment device 16 is supplied to a water mixing reservoir 18. The water mixing reservoir 18 also receives carbon dioxide that is extracted from the exhaust flue gas of the electrical generator plant 14 by a gas scrubber 20. The mixture of nutrient rich water and carbon dioxide is maintained at a pressure of approximately 2 psi over atmospheric pressure in order to maintain the mixture within the water mixing reservoir 18. The mixture of water and carbon dioxide is then supplied from the water mixing reservoir 18 to the algae growth facility 10 where the water is introduced to a plurality of algae growth troughs as will be described in greater detail.

The algae growth troughs are provided with an optimal amount of water, carbon dioxide, nutrient water or a combination of both, and can further be supplied with further carbon dioxide gas that is introduced directly into the algae growth troughs. In addition, outside air and an optimized amount of light is provided to each of the algae growth troughs for algae growth. During the photosynthesis process, the algae consumes the carbon dioxide and nutrients from the water as the algae cells multiply and grow within the algae growth troughs. The photosynthesis process produces both oxygen and some hydrogen which are captured from the algae growth troughs and can be introduced to a reformer 22 that supplies the oxygen rich gas to the electrical generator plant to improve the burn efficiency of the electrical generator plant 14 and can also supply a small portion of oxygen to the waste treatment plant 12 as needed in the waste treatment process. The oxygen can also be captured and processed for consumer use as a saleable byproduct.

As the algae grows within the troughs, the algae is regularly extracted from the troughs of the algae growth facility and the combined algae and water can be placed in a centrifuge 24 to separate the water from the algae. The water separated from the centrifuge is supplied to a water holding tank 26. The water from the water holding tank 26 can be UV treated by a UV treatment device 16 that can be the same as or different from the UV treatment device 16 discussed above. The algae mass separated by the centrifuge 24 can be utilized for extraction of oil for generation of biodiesel, bioethanol or other fuels can be used as feedstock, for production of other commercial products, or can be utilized as biofuel.

The algae that are extracted from the algae growth facility can also be introduced to an algae bioreactor 28 which can be supplied with nutrient rich water mixed with carbon dioxide and other added nutrients or can otherwise be treated in order to optimize the algae for algae growth and production of oil byproducts. By way of non-limiting example, some studies have shown that depriving algae of certain nutrients at certain stages of development can create certain desirable characteristics within the algae for optimum growth, optimum consumption of carbon dioxide or optimum production of hydrogen or oxygen byproducts. The algae from the bioreactor 28 are then reintroduced into the algae growth troughs of the algae growth facility 10. It should be noted that water from the waste treatment plant that has been UV treated by UV treatment device 16 can be filtered by a filter mechanism and supplied to a water holding tank 32 which can in turn be supplied to the electrical generator plant 14 as needed for plant operation. Furthermore, it should be noted that the water that is separated from the algae by the centrifuge 24 may be of sufficient quality and purity for release into the environment or can be reused by introduction into the water mixing reservoir 18.

The algae growth facility 10 in combination with a water treatment plant 12 and an electrical generation plant 14 or other industrial plants that similarly burn fossil fuels for production of material such as steel, glass, ceramics and other material all benefit from the integration of the algae growth facility and its ability to utilize nutrient rich water from the waste treatment plant as well as providing output water that is cleaner than the water received from the waste treatment plant and for capturing the carbon dioxide from the exhaust flue gas of the electrical generator plant or other industrial plant and using the carbon dioxide gas to facilitate algae growth that can be utilized for generation of fuels and other consumer products.

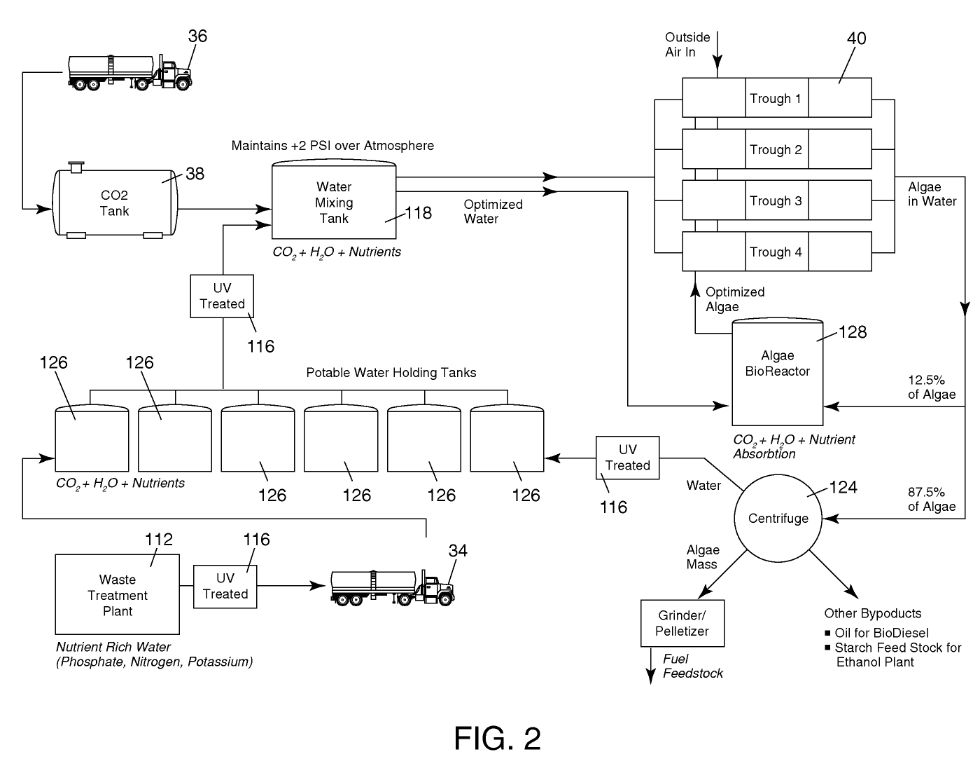

The flow diagram of FIG. 2 illustrates an alternative arrangement where an algae growth facility 110 is constructed geographically separate from a waste treatment plant 112 and an electrical generator plant or other industrial facility that has carbon dioxide rich exhaust flue gas. In this embodiment, water from the waste treatment plant is UV treated by a UV treatment device 116 and can be shipped by truck 34 or alternatively, by a train, boat, pipeline or other delivery method to the algae growth facility where the nutrient rich water can be stored in portable water tanks 126 and from there can be supplied to a water mixing tank 118 which mixes the nutrient rich water with carbon dioxide that can also be shipped in by truck 36 or other delivery method and stored in a carbon dioxide tank 38. The water supplied from the portable water holding tanks 126 can be UV treated by a UV treatment device 116 prior to introduction to the water mixing tank in order to kill any remaining or new bacteria that is formed in the portable water holding tanks 126. The mixture of water and carbon dioxide can be maintained at above atmospheric pressure within the water mixing tank 118 in order to maintain the mixture thereof.

The water can then be introduced to a plurality of algae growth troughs 40 as will be described in greater detail herein. The algae growth troughs 40 receive the combined water and carbon dioxide as well as optimized algae that are introduced at an introduction end of the troughs. In order to facilitate optimum growth of the algae within the troughs, lights are provided in the troughs or natural sunlight can also be utilized. The combination of algae and water is then extracted from the troughs 40 wherein a certain percentage of the algae can be reintroduced to the algae bioreactor 128 for reintroduction into the algae growth troughs 40 while a predetermined percentage of the combined algae in the water is introduced to the centrifuge 124 wherein the water is separated from the algae mass and the water is then returned to the portable water holding tanks 126 after being UV treated by a UV treatment device 116. The algae mass is then utilized for fuel, feed or other commercial products as discussed herein.

There is currently a need for substitute fuels that can replace fuels used in most aircraft and land vehicles. For defense security purposes it would be advantageous for this fuel not to be dependent on the availability of crude oil. For that reason, the study of alternative fuel sources is widespread. Biodiesel is one possible replacement for aircraft fuel. One of the largest potential sources of biodiesel is oil extracted from algae, a concept that has been extensively investigated and supported by the National Renewable Energy Lab. Another candidate fuel is one produced from coal using a liquefaction process. Although this coal-to-liquid process has been proven and refined over the past 70 years, it still has some giant hurdles to overcome. Specifically, the process generates enormous amounts of carbon dioxide and requires six to seven gallons of water to produce one gallon of fuel. Both are huge environmental hurdles that must be overcome if the coal-to-liquid process is to become a viable alternative.

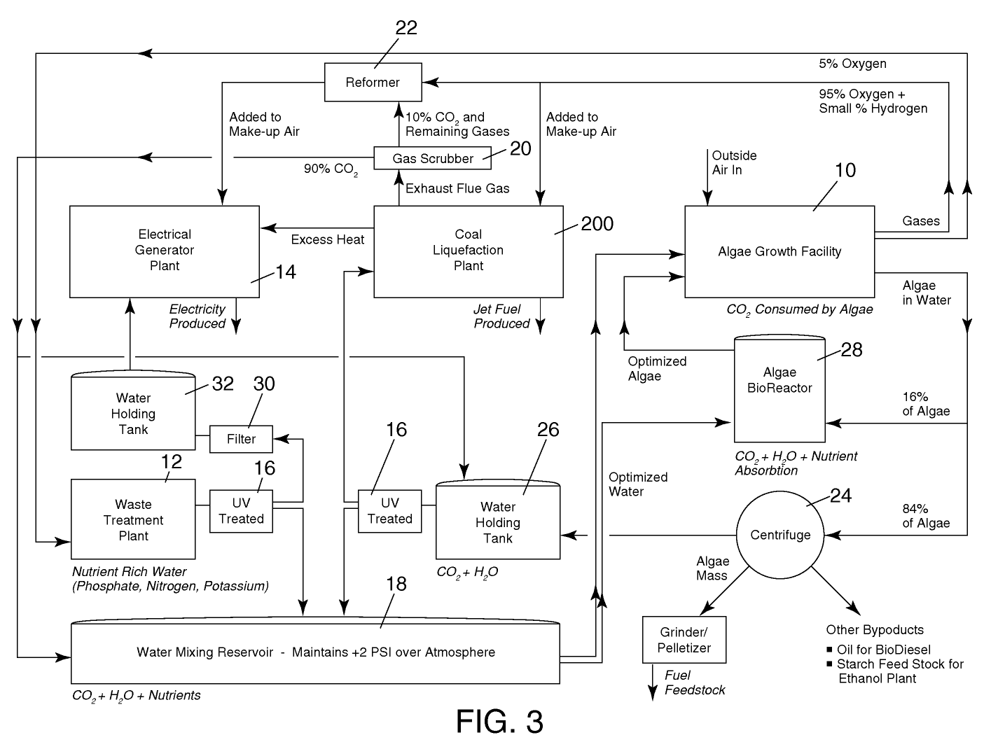

The integration of algae cultivation with the coal-to-liquid process can uniquely capture the carbon dioxide created by the coal-to-liquid process while the use of water from the algae growth facility in the coal liquefaction plant can satisfy the coal liquefaction plant water requirements. FIG. 3 shows a schematic diagram similar to FIG. 1, but integrating a coal liquefaction plant 200 along with the algae growth facility 10, waste treatment plant 12 and electrical generator plant 14. The coal liquefaction plant 200 provides exhaust flue gas having high carbon dioxide content which can be separated by a gas scrubber 20 so that a predetermined amount of the carbon dioxide is supplied to the water mixing reservoir 18 in addition to, or in place of, the carbon dioxide gas supplied by the electrical generator plant 14. It should be understood that the electrical generator plant 14 can be eliminated altogether as the coal liquefaction plant provides the desired carbon dioxide to the algae growth process. In the embodiment of FIG. 3, the oxygen gas harnessed from the algae growth facility 10 can be added to the coal liquefaction plant 200, as desired in the coal liquefaction process. The jet fuel produced from the coal liquefaction plant is therefore generated with reduced environmental impact as the carbon dioxide created by the coal liquefaction process can be harnessed and incorporated into the algae growth facility process.

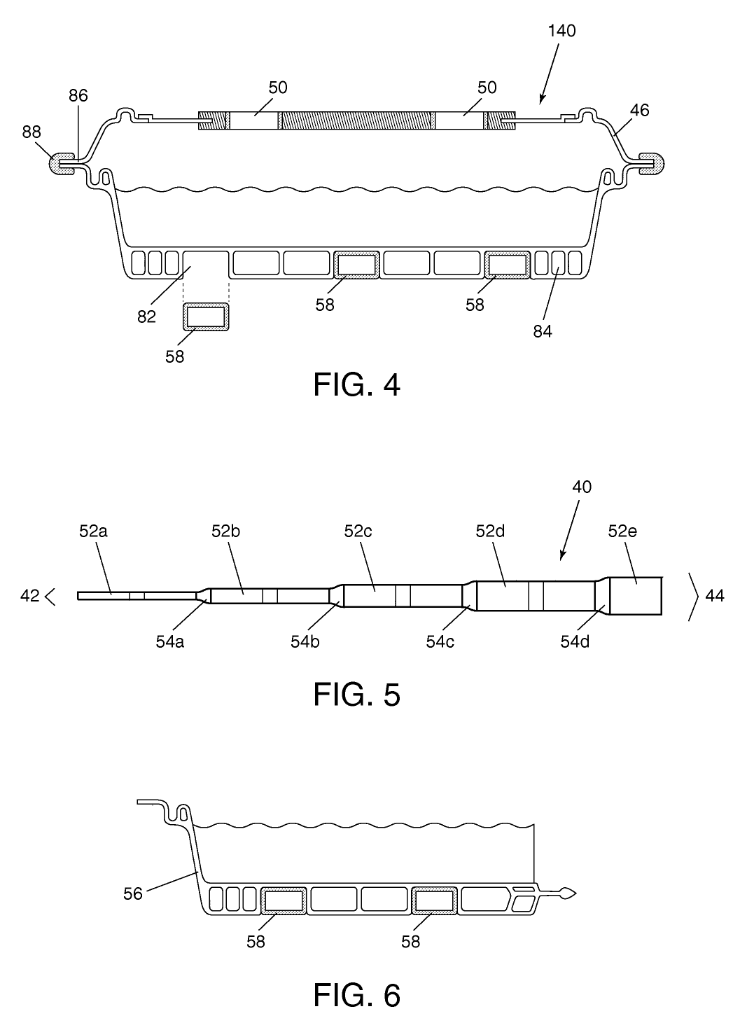

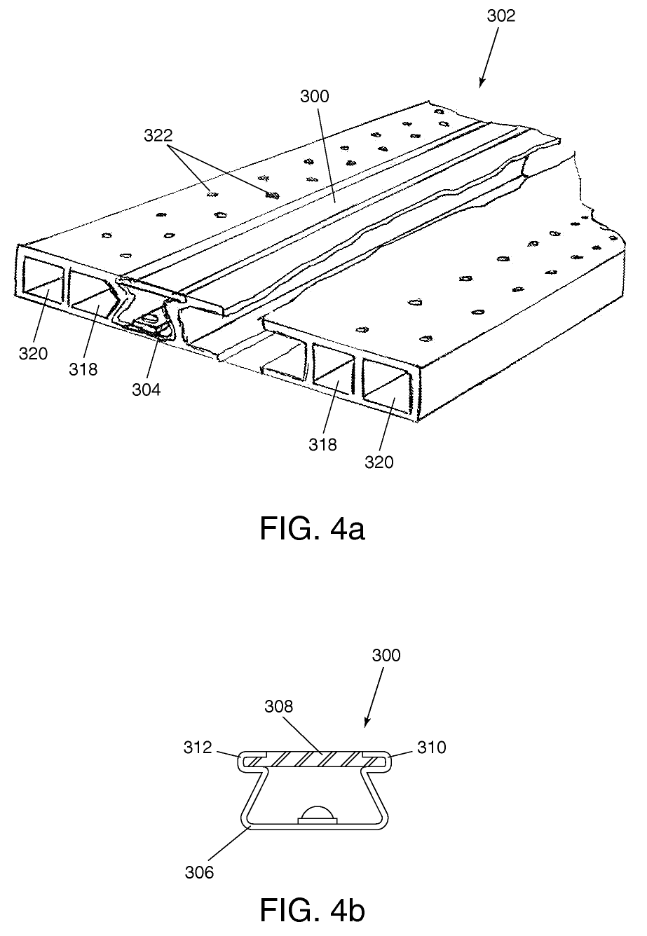

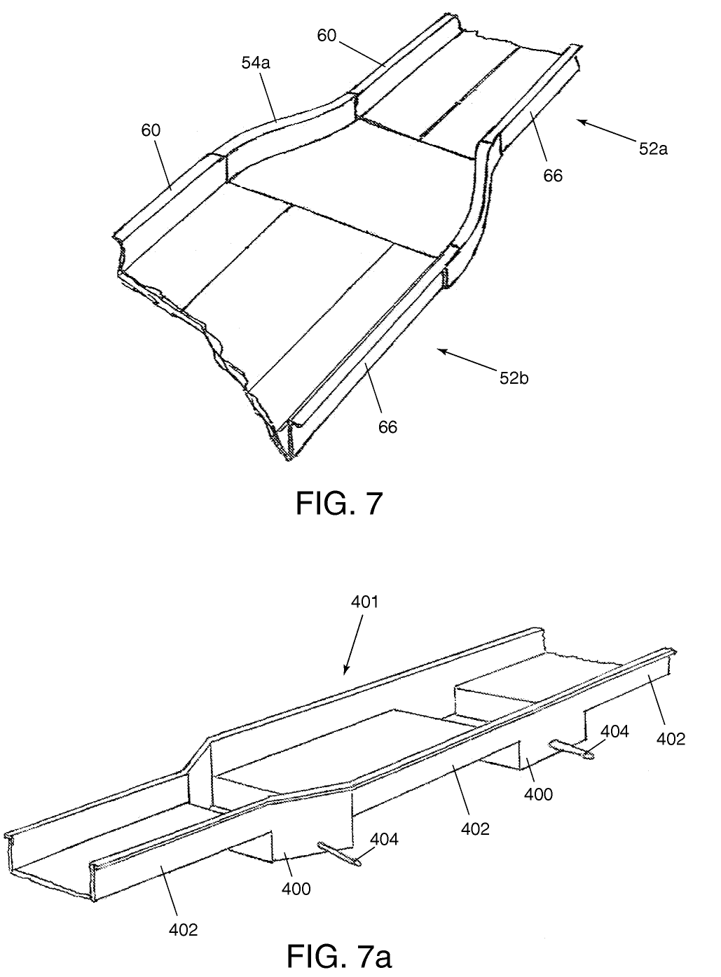

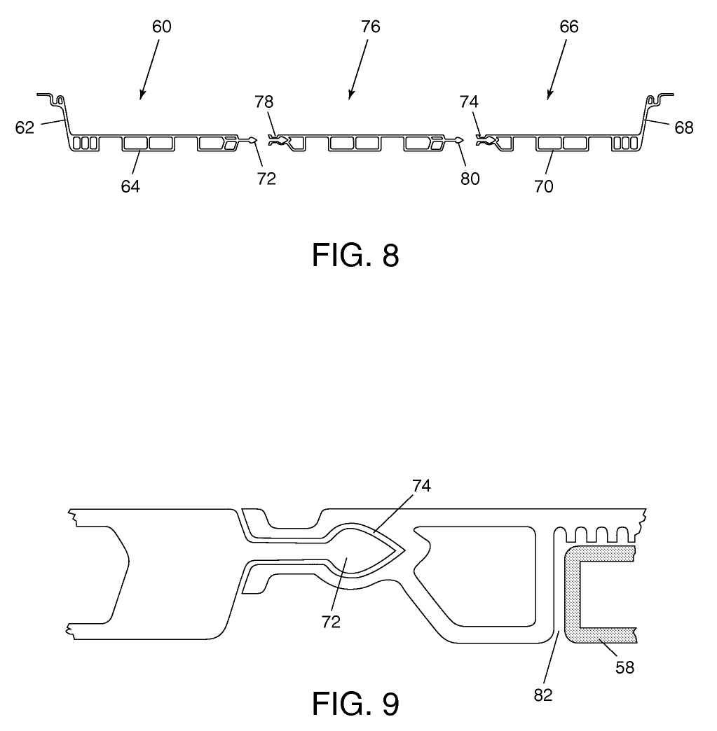

With reference to FIGS. 4-9, an exemplary algae growth trough 40, according to the principles of the present disclosure, will now be described. The algae growth trough 40 can have an algae introduction end 42 having a first width W1 and an algae extraction end 44 having a second width W2 wider than the first width W1. The trough 40 is provided with a cover 46 that can incorporate mounting features 48 for mounting a light source 50 for providing light inside of the trough. The cover 46 can also be made from a transparent material to allow light to travel therethrough in order to reduce or eliminate the necessity for a separate light source. An alternate light source from the bottom of the trough is shown in FIGS. 4a and 4b which illustrate a light strip 300 incorporated into a trough insert 302 which can be disposed in the bottom of the algae growth trough 40. The trough insert 302 can be formed with extruded aluminum or other material and can be fastened within the trough 40 by clamps, fasteners, or other known methods. The trough insert 302 can include a channel 304 for receiving the light strip 300. The light strip 300 can include a rolled or extruded metal housing 306 supporting a clear lens 308 between a pair of upper flanges 310, 312. A strip of lights 314 can be supported by a lower portion of the housing 306. The pair of upper flanges 310, 312 can be exposed to the water within the trough 40 to provide cooling of the light strip 300. The submerged light source can come from a LED variety of previously mentioned light sources, including piped light. The trough insert 302 can also include interior channels 318, 320 having vent holes 322 for allowing CO₂ and/or other nutrients to be supplied to the water within the troughs via the interior channels 318, 320. By pressuring the channels 318, 320, the CO₂ can be introduced without allowing water to fill the channels 318, 320. The troughs can be made at least in part from a polyethylene plastic, painted, or other material which resists the algae attaching itself to the walls and floor of the trough.

The troughs 40 include a plurality of straight sections 52a-52e each having progressively wider widths from the introduction end 42 to the extraction end 44. Each of the straight sections 52a-52e can have a generally constant width throughout and are attached to transition sections 54a-54d that widen from one end to another. Each of the straight sections 52a-52e can include extruded plastic sections 56 which can be reinforced by extruded metal and coated in a secondary operation or rolled metal sections either pre-coated or coated in a secondary operation 58, as shown in FIGS. 6, 7, 8, and 9. The straight sections 52a-52e can be assembled from a first side panel 60 including a sidewall portion 62 and a first floor portion 64. A second side panel 66 can be provided including a sidewall portion 68 and a second floor portion 70. The first floor portion and the second floor portion each include mutually engaging portions 72, 74, respectively, for sealingly engaging the first and second side panels 60, 66 together. In particular, the mutual engaging portion 72 of the first side panel 60 can include a protruding member that is received within the second mutual engaging portion 74 which defines a slot. Once the protruding member 72 is inserted into the slot 74, the first and second side panels can be welded together such as by laser or sonic welding to provide a sealed connection therebetween, as illustrated in FIG. 9. Additional sealing features such as gaskets and sealants can be utilized to enhance the seal obtained therebetween.

For providing straight sections having a wider width, additional center floor sections 76 can be provided including mutual engaging edge portions in the form of a slot 78 designed to receive the projecting portion 72 of the first side panel 60 and along a second edge, a protruding portion 80, which is designed to be received in the slot 74 of the second side panel 66. Again, the connection between the center floor section 76 and the side panels 60, 66 can be welded by sonic welding, laser welding, or other known bonding processes. Furthermore, the protruding portions 72, 80 can be provided with a bulbous end or other locking features while the slots 74, 78 can be provided with corresponding cavities for receiving the bulbous end of the protruding members 76 in a locking engagement.

The extruded panels 56 can include recessed cavities 82 in a bottom surface thereof for receiving the extruded metal reinforcement 58 such as extruded aluminum beams as shown in FIG. 4. Furthermore, the extruded panels 56 can include hollow channels 84 that provide added structural strength to the trough 40. The channels 84 can also be utilized for delivery of additional carbon dioxide gas to the trough, by providing additional apertures communicating between the channel and the surface of the trough and pressurizing the channels 84 with carbon dioxide gas, thus, causing the carbon dioxide to bubble through the openings in the panel and into the water. By pressurizing the channels 84 with carbon dioxide gas, water is prevented from leaking back into the channels.

The first and second side panels 60, 66 can each be provided with a side flange 86, and the cover 46 can be clamped to the side flanges 86 by clamp members 88. With the modular arrangement of the trough 40 as disclosed in FIGS. 4-6, 7, 8, and 9, the straight sections of 52a-52e of the trough 40 can be assembled using common extruded panel members with the addition of center floor sections for providing wider widths to the straight sections. Thus, the assembly of the troughs 40 can be greatly simplified. It should be noted that the transition sections 54a-54d can also be assembled in a manner similar to the straight wall sections, with the side panels having the tapered wall sections on each side and with varying length center panels or multiple center panels being utilized for providing varying widths to the transition sections 54a-54d. The trough 40 can also be constructed without straight sections, but with steadily increasing sidewalls along the entire length of the trough.

With reference to FIG. 7a, the trough 40′ can be provided with well portions 400 that extend below intermediate trough sections 402. The well portions 400 can include drain connections 404 to allow water to be extracted from the trough 40′ without disturbing the algae floating on the surface. Additional connections can be provided for introducing nutrient rich water into the well portions 400.

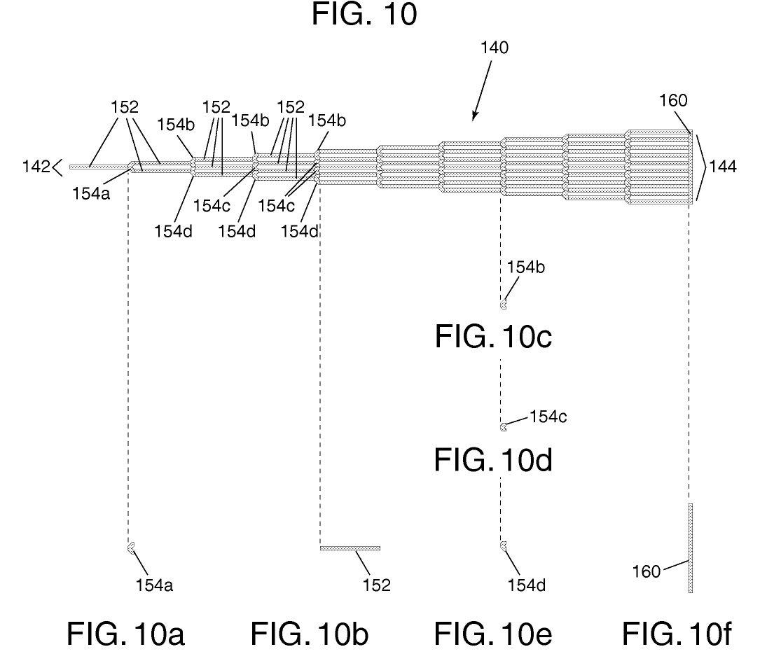

As shown in FIGS. 10-10F, an alternative trough assembly 140 can be utilized. The alternative trough assembly 140 includes an algae introduction end 142 and an algae extraction end 144 which can be provided with a cover and light source similar to the cover 146 and light source 50 as described above with respect to FIGS. 4-9. The trough assembly 140 can be made up of straight trough sections 152 which are connected to various style diverter section 154a-154d such that at the algae introduction end 142, a single straight trough section 152 is provided and is in communication with a diverter 154a which diverts flow to two parallel trough sections 52 which each terminate in left and right diverter sections 154b, 154d which divert flow to three parallel trough sections 152 which terminate in diverter sections including left and right diverter sections 154b, 154d and center diverter section 154c which divert flow to four trough sections 152 which each terminate in four diverter sections, including left diverter section 154b, right diverter section 154d, and two center diverter sections 154c. The diverter sections 154b, 154c, 154d divert flow into six straight trough sections 152 which terminate in five diverters including a left diverter 154b, a right diverter 154d, and three center diverters 154c. The diverters 154b, 154c, 154d each divert flow to eight different trough sections 152 which terminate at six diverter sections, including left diverter section 154b, right diverter section 154d, and four center diverter sections 154c. This pattern continues with an increase of one diverter at each junction and the increase of two troughs for each straight section until the extraction end 144 of the trough 140. According to the trough design 140, the entire trough structure can be assembled from common straight trough sections 152 and a variety of four different diverter sections used in combination in the manner described above to provide a trough structure 140 that increasingly widens from the algae introduction end 142 to the algae extraction end 144 to facilitate the growth of the algae. An end plate 160 can be provided for closing off the trough sections 152 at the extraction end 144 of the trough 140.

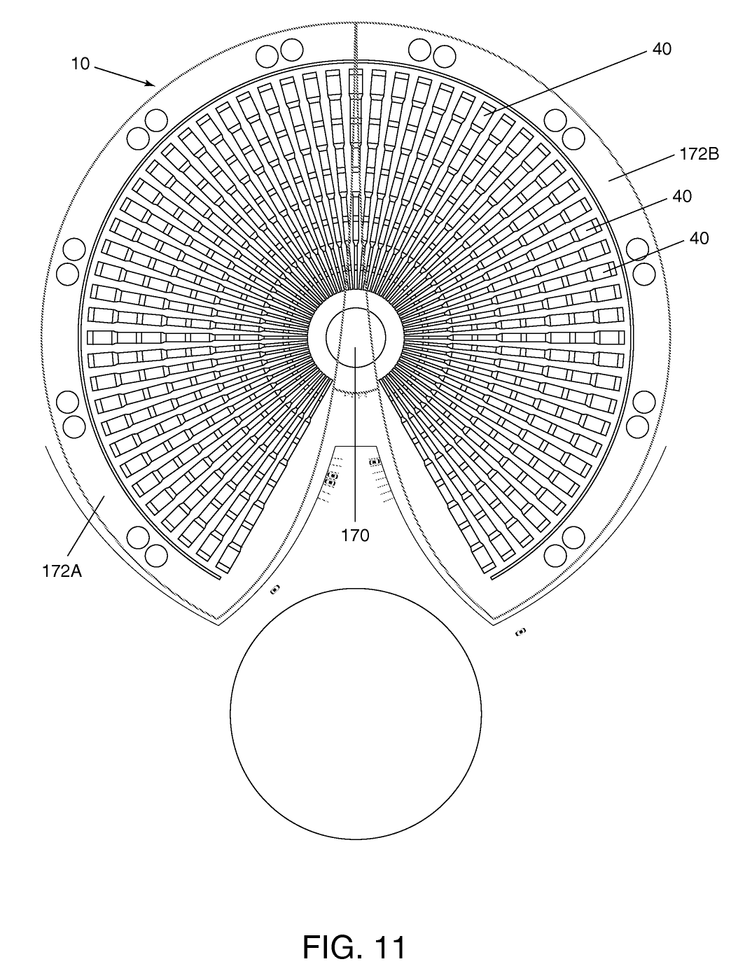

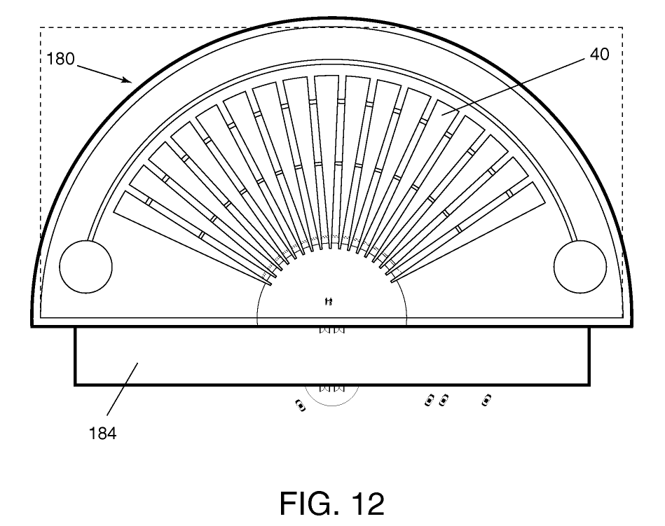

With reference to FIG. 11, a sample arrangement of an algae growth facility 10 is shown including a plurality of algae growth troughs 40 extending radially outward from a center section 170. The troughs 40 can be of the construction shown in FIGS. 4-9, or FIG. 10, or an alternative construction as desired. As illustrated in FIG. 11, a double fan arrangement 172a, 172b of the troughs 40 is provided within a circular algae growth facility 10. The algae growth facility can be provided with an office computer control center 174 for controlling the algae growth process, temperatures, and delivery of water and carbon dioxide to each of the troughs 40.

As shown in FIG. 12, a single fan arrangement is provided in a half-circle algae growth facility 180. An office/computer control center 184 is provided adjacent to the facility.

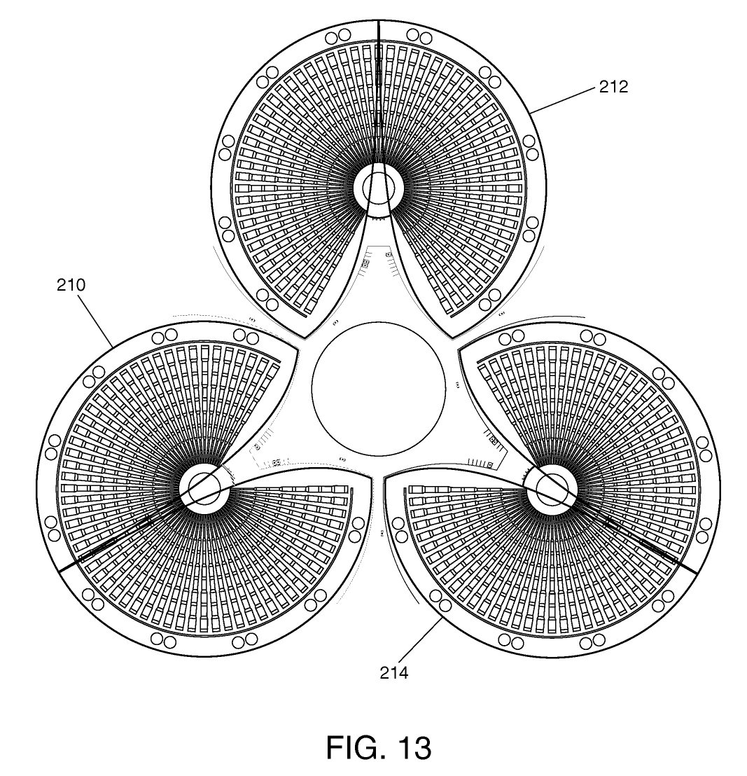

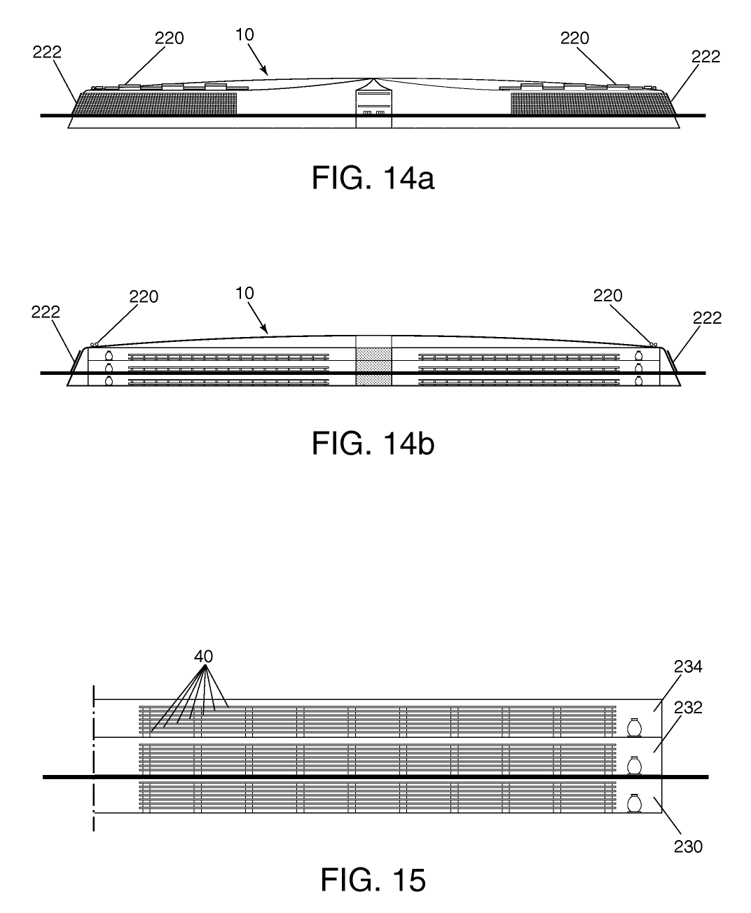

As still an additional alternative, multiple lobes 210, 212, 214 of algae growth facility can be utilized in combination with a center office facility for operating each of the lobes 210, 212, 214, as shown in FIG. 13. FIG. 14 shows a plan view of an exemplary algae growth facility 10 from the exterior view which can include air turbines 220 and solar panels 222 mounted to the outside walls and roof of the structure while the solar panels 222 and wind turbines 220 can be used to generate electricity that can supplement additional energy resources provided for operating the algae growth facility 10.

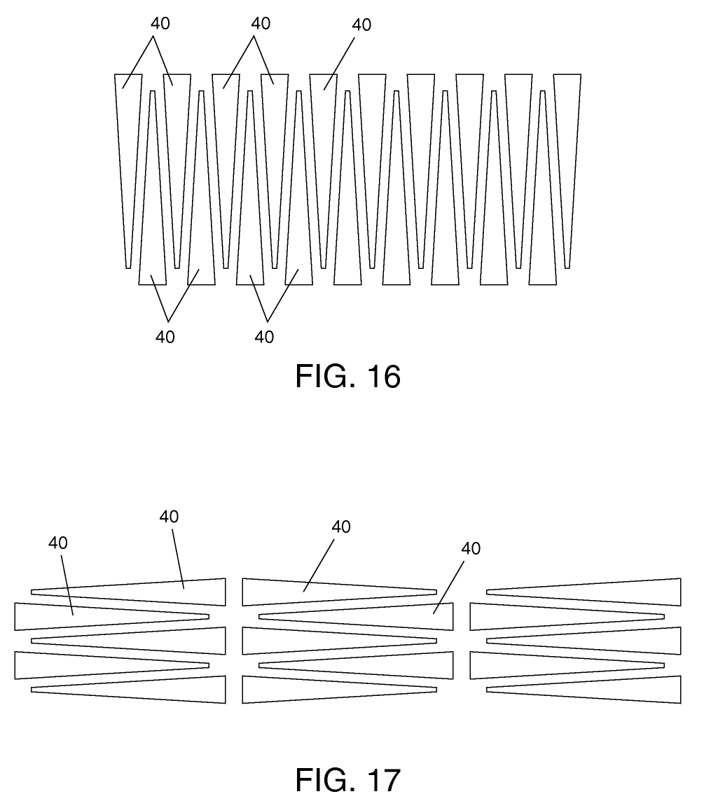

FIG. 15 illustrates a multi-level facility including three floors 230, 232, 234 each including seven layers of algae growth troughs 40 which are stacked one above another in order to provide an economic use of the available space for mass production of algae. It should be understood that one or more stacked layers of algae growth troughs 40 can be utilized for efficient use of facility space.

With respect to FIG. 16, an alternative arrangement of algae growth troughs 40 is shown wherein every other algae growth trough 40 is provided with its introduction end at opposite ends of the alignment. FIG. 17 further discloses another alternative arrangement of the algae growth troughs 40 according to the principles of the present disclosure.

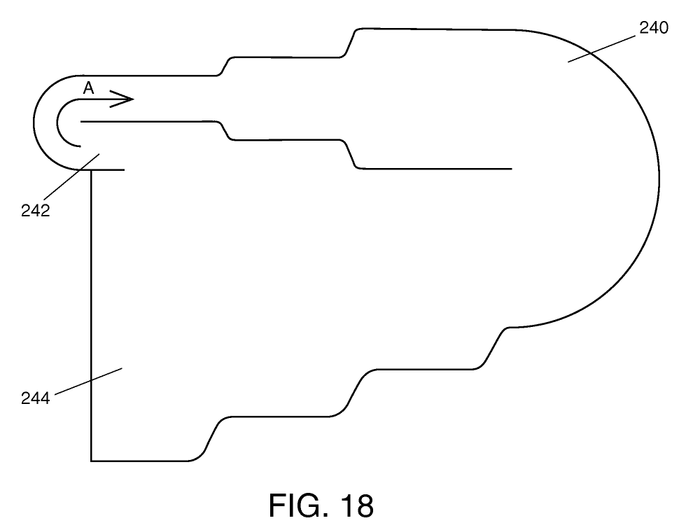

FIG. 18 provides yet another alternative arrangement of an algae growth trough 240 wherein the introduction end 242 of the algae growth trough is self-feeding from the extraction end 244 of the algae growth trough so that no additional algae need be reintroduced to the algae growth trough 240. In other words, as algae is initially introduced into the introduction end 242 of the algae growth trough, the algae grows in the direction of arrow A and travels along the trough from its narrow introduction end 242 toward the extraction end 244. Additional water can be introduced at the introduction end 242, but as algae approaches the extraction end 244 of the trough, some algae passes back into algae introduction end 242 which mixes with new water being introduced into the trough 240 and then travels through the trough 240 from its narrow end 242 to its wider extraction end 244. Thus, it becomes unnecessary to manually provide additional algae into the system as the algae growth trough 240 is self-feeding.

Approximately 1000 tons of coal can be consumed each day in the process of operating a 100 MW electrical power plant. One feature of the present disclosure is an almost unlimited water supply using return UV purified water from a city waste treatment plant. The waste treatment plant also provides all the required nutrients for growing algae. Each ton of coal produces three tons of carbon dioxide for a total of 3000 tons of carbon dioxide each day. It takes about 2.5 tons of carbon dioxide to produce one ton of algae. The oil content of this amount of algae can produce 120 tons or 30 thousand gallons per day of biodiesel fuel wherein one ton of liquid oil yields 250 gallons of fuel. The carbohydrate content of this amount of algae can produce 180 tons or 45 thousand gallons per day of ethanol. With this type of production, a daily total production capacity can exceed 75 thousand gallons of biofuel and on an annual basis, 27,375,000 gallons of biofuel will be produced.

According to calculated simulations, each trough can be five feet wide and 420 feet long with continuous harvesting, 24 hours per day, seven days a week. Each harvest will produce 32 pounds of algae per square foot, thus yielding 16.8 tons of algae, wherein one ton of algae can consume 2.5 tons of carbon dioxide. Therefore, each trough will consume 16.8 tons of algae×2.5 tons of carbon dioxide=42 tons of carbon dioxide. A typical 100 MW electrical plant consumes 1000 tons of coal each day and produces 3000 tons of carbon dioxide each day. 3000 tons divided by 42 tons per trough equals 71 troughs per 100 MW of power at 100% efficiency. An efficiency operation of 85% would require 61 troughs. Algae provides a triple growth rate per day such that the introduction of 0.0027 pounds per square foot after nine days will yield 53.1441 pounds based upon the initial introduction of 0.0027 pounds of algae. If the algae goes into the expansion trough at 5.9 pounds, the algae yield after 24 hours equals 17.74 pounds, and after 38 hours equals 38 pounds. Of the 38 pound yield, six pounds can be recycled and added at the introduction end of the trough while 32 pounds of yield can be utilized with the throughput being every two days for one complete cycle. Thus, the algae growth facility can be constructed in such a manner as to consume virtually all of the excess carbon dioxide of an electrical generator plant or other industrial plant which exhausts carbon dioxide flue gas. The use of municipal waste treatment plant water further provides a useful application of the waste water which otherwise will go through expensive treatment processing in order to be reintroduced into the environment.

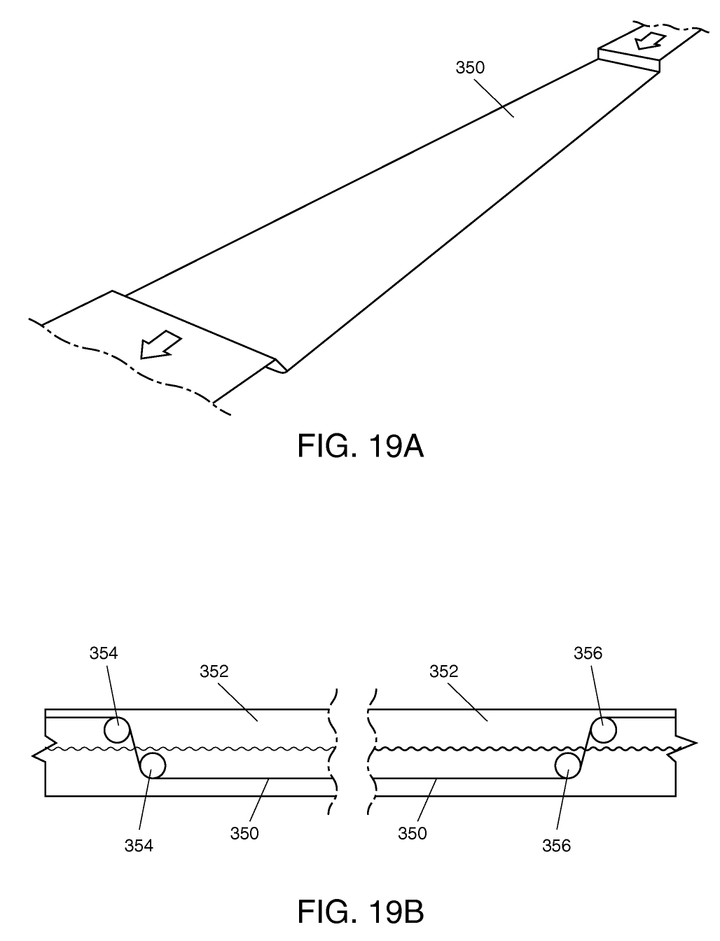

There are many different types of algae including Plantonic algae which is composed by single cell plants which float freely on the surface and derive their nutrients from the water flowing around them. Benthic algae is composed of single cell plants which generally live in close relationship with a submerged surface. Such organisms permanently attach to the submerged surface and derive their nutrients from the water flowing through them. As shown in FIGS. 19A and 19B, a conveyor matt 350 is shown for growing and harvesting benthic algae within an elongated trough 352 (FIG. 19B). The conveyor matt 350 can be a continuous loop-type matt on which the bentic algae attaches and is then scraped off or otherwise removed for harvesting. Alternatively, the matt 350 can be formed from an organic material that can be used to provide a substrate for growth of benthic algae and can then be subsequently treated and burned along with the algae as bio-fuel. The matt 350 can be supported by a plurality of rollers 354 at a first end and by a plurality of rollers 356 at a second end so that the matt 350 is disposed below a surface of the water in the trough 352. One or more plantonic algae strains can grow along side one or more benthic algae strains, with-in the same nutrient system. The combined algae growth systems relationship between plantonic and benthic algae growth strains may grow in combination with, or in a symbiotic relationship. A belt system is included to used as a carrier and harvesting platform for plantonic algae and an attachment platform for benthic algae. Multiple materials may be used for a reusable belt (ranging from metal to plastic) pre-coated with nutrients and a non-reuse, consumable belt. The reusable belt can be designed to retain residual algae to become the initial starter algae for the continuous algae growth cycle. The initial starter algae may then become pre-coated with nutrients to stimulate the continuous algae growth cycle. The non-reusable, and consumable belt, is one that is made of fibrous material (synthetic or natural) pre-coated or embedded with nutrients, that dissolves after a certain period of time. The non-reusable, and consumable belts are then processed directly with the algae.

What is claimed is:

1. A method of mass production of aquatic plants, comprising:
providing a plant growth trough having a plant introduction end having a first width and a plant extraction end having a second width wider than said first width;
supplying water to said trough from a water treatment facility;
introducing CO₂ from a combustion source to said water;
providing light to said trough;
introducing an aquatic plant to said introduction end of said trough; and
extracting said aquatic plants from said extraction end of said trough.

2. The method according to claim 1, wherein said CO₂ is supplied from an electrical generator plant

3. The method according to claim 1, wherein said light is supplied by LEDs emitting light in specific wave lengths.

4. The method according to claim 1, wherein said light is supplied by at least one of sunlight, LEDs, incandescent light.

5. The method according to claim 1, wherein said combustion source is an electrical coal generator plant.

6. The method according to claim 1, wherein said combustion source is a CO₂ emitting facility.

7. The method according to claim 1, wherein said combustion source is a manufacturing plant using combustion in a manufacturing process

8. The method according to claim 1, wherein said combustion source is a hydrocarbon fuel source.

9. The method according to claim 1, wherein said water is treated with a UV light to kill bacteria therein.

10. The method according to claim 1, wherein said extracted aquatic plants are treated with de-watering equipment to separate water from said aquatic plants.

11. The method according to claim 6, wherein oxygen is extracted from said trough for supplying to said electrical generator plant.

12. The method according to claim 1, wherein said step of introducing CO₂ includes introducing CO₂ directly into water in said plant growth trough.

13. The method according to claim 1, wherein said step of providing light to said trough includes providing a light submerged in said water in said trough.

14. The method according to claim 1, wherein said step of extracting said aquatic plants includes withdrawing a conveyor matt from said trough including aquatic plants attached thereto.

15. The method according to claim 1, wherein said trough includes at least one well portion that is deeper than a remainder of said trough to allow water to be extracted from the well portion without disturbing the algae on the surface.

16. A system for mass production of algae, comprising:
a plurality of troughs each having an algae introduction end having a first width and an algae extraction end having a second width wider than said first width, said plurality of troughs each being provided with a cover and a light source for providing light inside of said trough;
a source of water attached to said plurality of troughs, said source of water including a water treatment plant; and
a source of CO₂ in communication with at least one of said plurality of troughs and said source of water, said source of CO₂ including a combustion exhaust gas from one of an electricity generation plant, a steam generation plant and an industrial manufacturing plant.

17. The system according to claim 16, wherein said troughs are made at least in part from a polyethylene plastic.

18. The system according to claim 16, wherein said troughs include a plurality of straight sections, each having a generally constant width, attached to transition sections that widen from one end to another.

19. The system according to claim 16, wherein each of said straight sections have a same width with said transition section diverting flow to two adjacent straight sections.

20. The system according to claim 16, wherein each of said straight sections include extruded plastic sections.

21. The system according to claim 16, wherein said extruded plastic sections are reinforced by metal sections.

22. The system according to claim 16, wherein said straight sections are assembled from a first side panel including a side wall portion and a first floor portion and a second side panel including a side wall portion and a second floor portion.

23. The system according to claim 22, wherein said first floor portion and said second floor portion each include mutual engaging portions for sealingly engaging said first and second side panels together.

24. The system according to claim 23, wherein said first floor portion and said second floor portion are welded to one another.

25. The system according to claim 22, further comprising a center floor section including first and second edge portions adapted to engage respective ones of said first floor portion and said second floor portion.

26. The system according to claim 25, wherein said first floor portion is welded to said center floor section and said second floor portion is welded to said center floor section.

27. The system according to claim 16, further comprising a channel in said plurality of troughs for introducing CO₂ into said water.

28. The system according to claim 16, further comprising a light source in said plurality of troughs.

29. The system according to claim 16, further comprising a conveyor matt disposed in said trough for traversing from said algae introduction end to said algae extraction end.

30. The system according to claim 16, wherein said trough includes at least one well portion that is deeper than a remainder of said trough and a drain disposed in said well portion.

31. A system for mass production of algae, comprising:
a trough having an algae introduction end having a first width and an algae extraction end having a second width wider than said first width, said trough being provided with a light source for providing light inside of said trough;
a source of water attached to said plurality of troughs, said source of water including a water treatment plant; and
a source of CO₂ in communication with at least one of said trough and said source of water, said source of CO₂ including a combustion exhaust gas from one of an electricity generation plant, a steam generation plant and an industrial manufacturing plant.

32. A system for mass production of algae, comprising:
a trough having an algae introduction end and an algae extraction end, said trough being provided with a light source for providing light inside of said trough;
a source of water attached to said plurality of troughs; and
a source of CO₂ in communication with at least one of said trough and said source of water, said source of CO₂ including a combustion exhaust gas from one of an electricity generation plant, a steam generation plant and an industrial manufacturing plant.

33. A system for mass production of algae, comprising:
a trough having an algae introduction end and an algae extraction end;
a source of water attached to said plurality of troughs, said source of water including a water treatment plant; and
a source of CO₂ in communication with at least one of said trough and said source of water, said source of CO₂ including a combustion exhaust gas from one of an electricity generation plant, a steam generation plant and an industrial manufacturing plant.
